(19) United States
(12) Patent Application Publication
(10) Pub. No.: US 2019/0053778 A1
(43) Pub. Date: Feb. 21, 2019

(54) AUSCULTATION DEVICE WITH MODULAR CHEST PIECE AND ECG MODULE

(71) Applicant: VITALCHAINS CORPORATION, Menlo Park, CA (US)

(72) Inventor: TZU-CHIH LIN, Hsinchu (TW)

(21) Appl. No.: 16/104,147

(22) Filed: Aug. 17, 2018

Related U.S. Application Data
(60) Provisional application No. 62/546,694, filed on Aug. 17, 2017.

(51) Int. Cl. A61B 7/04; A61B 5/00; A61B 5/0408; A61B 5/0205

(52) U.S. Cl. CPC A61B 7/04; A61B 5/0002; A61B 2560/0443; A61B 5/0205; A61B 5/0408

(57) ABSTRACT

The present disclosure provides an auscultation device. The auscultation device comprises a sound capturing element, a chest piece holder, and a connecting structure. The chest piece holder is disposed on a distal end of the auscultation device, coupled to the sound capturing element, and includes a connecting structure. The chest piece comprises a dislocating structure removably coupled to the connecting structure of the chest piece holder.

### CROSS-REFERENCE TO RELATED APPLICATIONS

This application claims the benefit of U.S. provisional patent application No. 62/546,694, filed on Aug. 17, 2017, which is hereby incorporated by reference in its entirety.

### FIELD

The invention pertains to the field of auscultation. More particularly, the invention pertains to auscultation devices with modular chest piece that can he replaceably removed.

### BACKGROUND

The heart and body produce hundreds of specific sounds, including heart, king, bowel, circulatory system, and Korotkoff sounds. These sounds and combinations thereof are indicative of normal and abnormal conditions. Knowledge of these sounds provides valuable diagnostic information to the physician. The art of listening to, these sounds and using them as diagnostic aids is known as auscultation. The tool that physicians use to perform auscultation is referred to as a ausculation device.

The design of auscultation device has not changed much since the original invention of auscultation devices by the French physician Rene Laennec about 200 years ago. The auscultation device has been the main diagnostic tool for generations of physicians and other health practitioners. However the sound collected by conventional auscultation device cannot be easily recorded, captured, digitized, and analyzed. The physicians would need to analyze the sound instantly while the sound is received by the conventional auscultation device.

Recently, a number of digital auscultation devices have finally found their way to the market. The sound of auscultation can be recorded, captured, digitized, or analyzed by commercially available digital auscultation devices. Each of the commercially available digital auscultation devices, or conventional digital ausculation devices, is generally consisted of a chest piece and a diaphragm to contact the body and collect the sounds, an acoustic sensor to capture the sounds collected by the chest piece and the diaphragm, and a control module to digitize, analyze, or record the sounds captured by the microphone. These digital auscultation devices may be configured to detect Korotkoff sounds, or sounds from heart, lung, bowel, or circulatory system.

However, most commercially available digital auscultation devices are designed to be coupled to one specific signal input component The signal input component can be an Y-tube with ear pieces, an audio output jack, a phone jack, or a wireless signal transmission component. They do not offer much flexibility to allow modular addition or integration of other functionalities. The application and function of the conventional digital auscultation device are thus limited by the single-output design.

Additionally, different chest pieces may be required when auscultating for sounds generated by different organs or by different subjects, such as chest piece for adults or for children. Nevertheless, the chest pieces in both the conventional auscultation devices or the digital auscultation devices are not interchangeable. After using a conventional auscultation device or a digital auscultation device on one subject, the user would have to switch to another auscultation device when an auscultation subject or a target organ is changed.

Furthermore, a major setback when changing the chest pieces is exposing the acoustic sensor in the digital auscultation device to the surrounding environment. Because the acoustic sensor in the digital auscultation device is not shielded by the chest piece during the changeover, the acoustic sensor may be prone to damage. Another setback when changing the chest piece is the scrubbing sound or noise caused by the changeover. The user may unintentionally change the chest piece without realizing one is still wearing an earpiece of a Y-tube coupled with the digital auscultation device, in this case, the noise generated will be magnified and cause irritation to the user.

Therefore, it is desirable to provide an improved auscultation device with an interchangeable chest piece.

It is also desirable to provide an in auscultation device design that are modular and capable of integrating other signal input components.

It is also desirable to provide an improved auscultation device that blocks the sound transmission route and shield the acoustic sensor during the chest piece changeover.

### SUMMARY OF THE INVENTION

In view of the shortcomings in the art, it is an object of the present invention to provide a modular auscultation device with interchangeable chest pieces or diaphragms having varying sizes, geometry, material, and other structural configurations that may be beneficial to the operation or performance of the auscultation device under different circumstances.

It is another object of the present invention to provide a modular digital auscultation device that is capable of working with and operatively connecting to a variety of signal input components.

It is another object of the invention to provide a modular auscultation device capable of working with additional sensor module.

It is another object of the invention to provide a protective mechanism for the acoustic sensor during the changeover of the chest pieces.

An embodiment of the present disclosure provides an auscultation device. The auscultation device comprises a sound capturing element, a chest piece holder disposed on a distal end of the auscultation device, coupled to the sound capturing element, and comprising a connecting structure and a chest piece comprising a dislocating structure removably coupled to the connecting structure of the chest piece holder.

In a preferred embodiment, the dislocating structure comprises a plurality of projections. The connecting structure comprise a flange having a plurality of notches, a plurality of wings, and a sidewall. The notches and the wings are alternatingly arranged around the flange. The notches and the sidewall form a plurality of notch receiving spaces, and the wings and the sidewall form a plurality of wing receiving spaces.

In a preferred embodiment, each of the projection of the dislocating structure being received by each of the notch receiving spaces of the flange corresponds a first state of the auscultation device.

In a preferred embodiment, each of the projections of the dislocating structure being at least partially received by each of the wing receiving spaces of the flange corresponds a second state of the auscultation device.

In a preferred embodiment, the auscultation device further comprises a partition plate between the sound capturing element and the chest piece holder. The partition plate comprises a central hole and a pivot.

In a preferred embodiment, the auscultation device further comprises a switching piece between the partition plate and the chest piece holder. The switching piece comprises a door being rotatable about the pivot.

In a preferred embodiment, the door of the switching piece is not blocking the central hole of the partition plate, corresponding to a third state of the switching piece and the partition plate.

In a preferred embodiment, the door of the switching piece is at least partially blocking the central hole of the partition plate, corresponding to a fourth state of switching piece and the partition plate.

In a preferred embodiment, the switching piece further comprises a protrusion rotatable about the pivot. The chest piece further comprises a ditch, and the protrusion is m contact with the chest piece through the ditch.

In a preferred embodiment, the digital auscultation device is in the third state when the protrusion of the switching piece is at a first position.

In preferred embodiment, the digital auscultation device is in the fourth state when the protrusion of the switching piece is at a second position.

In a preferred embodiment, the auscultation device is in the first state when the switching piece and the partition plate are in the fourth state for at least partially blocking a sound transmission route from passing through the central hole to the microphone. A rotation of the switching piece is triggered by a rotation of the chest piece through the protrusion and transforms the switching piece and the partition plate from the fourth state into the third state. The auscultation device is in the second state when the switching piece and the partition plate are in the third state for unblocking the sound transmission route from passing through the central hole to the sound capturing element.

In a preferred embodiment, the auscultation device further comprises a diaphragm, and the diaphragm can be of different sizes.

In a preferred embodiment, the chest piece further comprises an extended surface not covered by the diaphragm, and one or more ECG plates disposed on the extended surface.

In a preferred embodiment, the auscultation device further comprises a housing having a digital part and a proximal part, a control module in the housing, a microphone coupled to the control module, and an output module connectable with a signal input component and disposed on the proximal part of the housing.

In a preferred embodiment, the output module comprises a first latch, a first pogo pin and a first male screw thread for connecting with different signal input components.

In a preferred embodiment, each of the different signal input component comprises a first female screw thread, a first pogo pin connector, and a first latch groove.

In a preferred embodiment, the output module comprises a second latch groove, a second pogo pin connector, and a second female screw thread for connecting with different signal input components.

In a preferred embodiment, each of the different signal input component comprises, a second male screw thread, a second pogo pin connector, and a second latch.

In a preferred embodiment, the digital auscultation device further comprises a rotary clasp for connecting the signal output and the output module.

An embodiment of the present disclosure provides a digital auscultation device. The digital auscultation device comprises a housing having a digital part and a proximal part, a control module in the housing, a microphone coupled to the control module, an output module connectable with an signal input component and disposed on the proximal part of the housing, a chest piece holder disposed on the distal part of the housing and comprising a connecting structure, a chest piece comprising a dislocating structure removably coupled to the connecting structure of the chest piece holder, and a diaphragm removably coupled to the chest piece.

### BRIEF DESCRIPTION OF THE DRAWINGS

Implementations of the present technology will not be described, by way of example only, with reference to the attached figures.

FIG. 1 is a perspective view of a digital auscultation device in accordance with an exemplary embodiment of the present disclosure.

FIG. 2 is another perspective view of the digital auscultation device in accordance with an exemplary embodiment of the present disclosure.

FIG. 3 is an exploded view of the digital auscultation device in accordance with an exemplary embodiment of the present disclosure.

FIGS. 4A and 4B are front side and rear side views of a chest piece and a chest piece holder; FIG. 4C is a perspective view of the chest piece and the chest piece holder; FIGS. 4D and 4E are cross-sectional views of the chest piece holder, in accordance with an exemplary embodiment of the present disclosure.

FIGS. 5A and 5B are perspective views of the chest piece and the chest piece holder different states, in accordance with an exemplary embodiment of the present, disclosure.

FIGS. 6A and 6B are exploded views of another digital auscultation device, in accordance with an exemplary embodiment of the present disclosure.

FIG. 7 is an exploded view of another digital auscultation device, in accordance with an exemplary embodiment of the present disclosure.

FIGS. 8A and 8B are perspective views of a partition plate and a switching piece in different states, in accordance with an exemplary embodiment of the present disclosure.

FIGS. 9A and 9B are exploded views of the digital auscultation device in different states; FIGS. 9C and 9D are perspective views of the digital auscultation device in different states, in accordance with an exemplary embodiment of the present disclosure.

FIG. 10 is an exploded view of another digital auscultation device, in accordance with an exemplar embodiment of the present disclosure.

FIGS. 11A and 11B are perspective views of another digital auscultation device, in accordance with an exemplary embodiment of the present disclosure.

FIG. 12 is an exploded view of the digital auscultation device of FIGS. 11A and 11B, in accordance with an exemplary embodiment of the present disclosure.

FIG. 13 is an exploded view of a non-digital auscultation device, in accordance with an exemplary embodiment of the present disclosure.

FIG. 14 is an exploded view of another non-digital auscultation device, in accordance with an exemplary embodiment of the present disclosure.

FIG. 15 is a perspective view on a proximal part of the digital auscultation device, in accordance with an exemplary embodiment of the present disclosure.

FIG. 16 is a perspective view of a signal input component and the proximal part of a digital auscultation device, in accordance with an exemplary embodiment of the present disclosure.

FIG. 17A-17C are perspective views of the signal input components, the digital auscultation devices, and a plurality of rotary clasps, in accordance with an exemplary embodiment of the present disclosure.

FIG. 18 is a perspective view of the rotary clasp and the signal input component in accordance with an exemplary embodiment of the present disclosure.

### DETAILED DESCRIPTION

The present disclosure will now be described more filly hereinafter with reference to the accompany drawings, in which exemplary embodiments of the disclosure are shown. This disclosure may, however, be embodied in many different forms and should not he construed as limited to the exemplary embodiments set forth herein. Rather, these exemplary embodiments are provided so that, this disclosure will be thorough and complete, and will fully convey the scope of the disclosure to those skilled in the art. Like reference numerals refer to like elements thought the present disclosure.

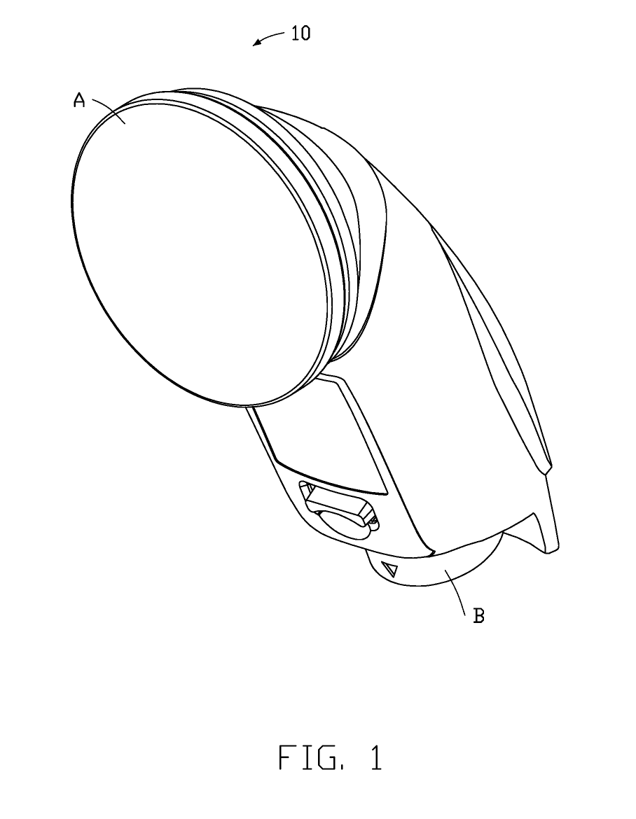

Referring to FIG. 1, a digital auscultation device 10 is provided in accordance with an exemplary embodiment of the present disclosure. The digital auscultation device 10 comprises a distal part A and a proximal part B. When using the digital auscultation device 10, the distal part A is closer to a surface of an auscultation subject, and the proximal part B is closer to a user. The tem “user” refers to the individual conducting auscultation on the subject. The uses may hold the proximal, part B to conduct auscultation, and one or more components on the distal part A may be in contact with the surface of the subject hen using the digital auscultation device 10.

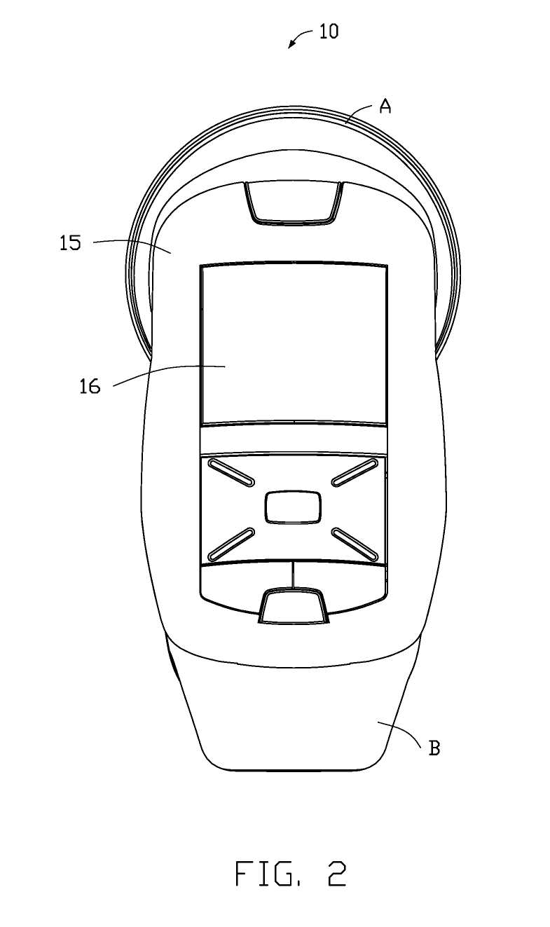

Referring to FIG. 2, an perspective view of the digital auscultation device 10 is provided in accordance with an exemplary embodiment of the present disclosure. A housing 15 generally forms an exterior of the digital auscultation device 10 and provides protection for interior components. A user interface 16 is on the exterior of the digital auscultation device 10, and may be located on a substantially middle location with respect to the distal part A and the proximal part B. The user interface 16 comprises one or more of screens or buttons, and the user may input commands to the digital auscultation device 10 through the user interface 16. The user may also receive instructions, advices, or auscultation results from the user interface 16.

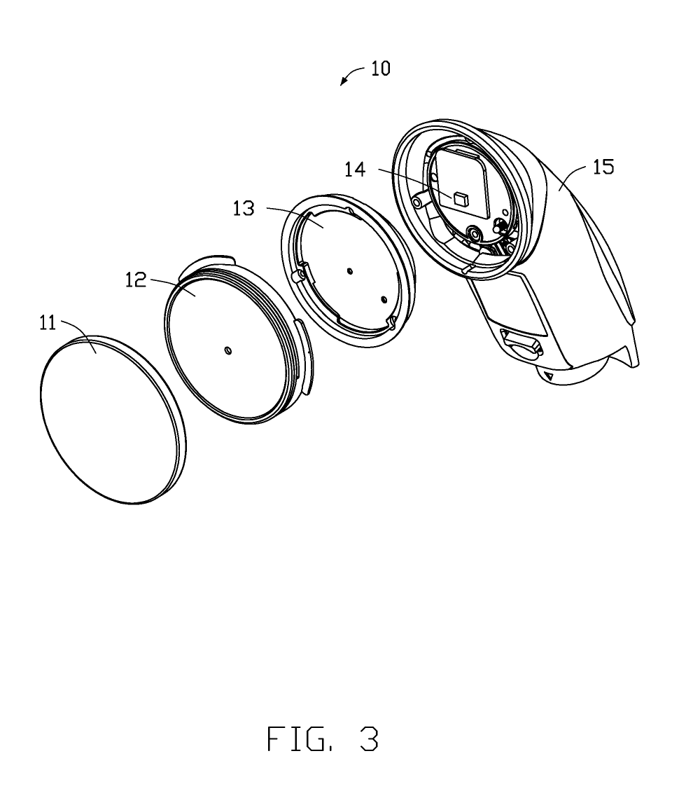

Referring to FIG. 3, an exploded view of the digital auscultation device 10 is provided in accordance with an exemplary embodiment of the present disclosure. A microphone 14 is inserted in the housing 15 and located on the distal part A. The microphone 14 is a sound capturing element in the digital auscultation device 10 that captures analog signals and transmit the analog signals to a control module (not shown) in the housing 15. The control module may receive instructions or commands from the user from the user interface 16, instruct the microphone 14 to capture one or more wavelength ranges of analog signals, receive the analog signals from the microphone 14, convert the analog signals into digital signals, record the digital signals, analyze the digital signals, and then transmit auscultation results to signal input components. The control module may comprise a converter for analog-digital conversion, a processor for analyzing or processing signals, a memory for storing data or instructions, and an 110 unit. A chest piece holder 13 is disposed on the distal part A for receiving a chest piece 12. The chest piece 12 is removably coupled to the chest piece holder 13. A diaphragm 11 is composed of a soft and flexible material, and is removably coupled onto the chest piece 12. A diameter of the diaphragm is about 30 to 50 mm. Preferably, the diameter of the diaphragm is 47 mm for auscultation of adult subjects, or 35 mm for pediatric auscultation. The diaphragm 11 and the chest piece 12 collect auscultation sounds from the surface of the subject when using the digital auscultation device 10. When conducting auscultation on different subjects, the user may change the diaphragm 11 between the subjects for hygiene.

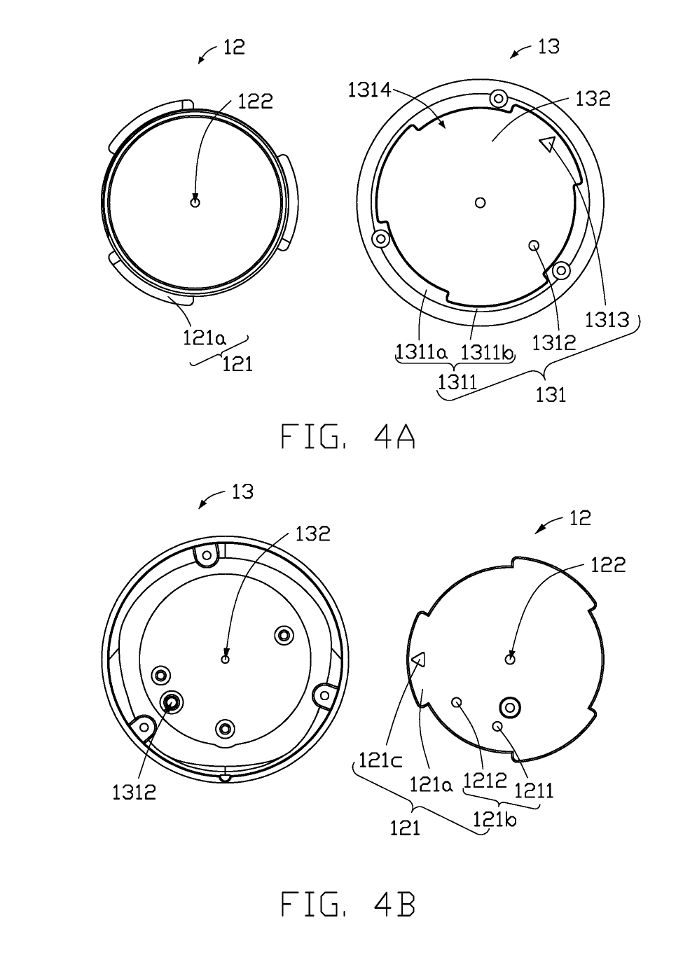

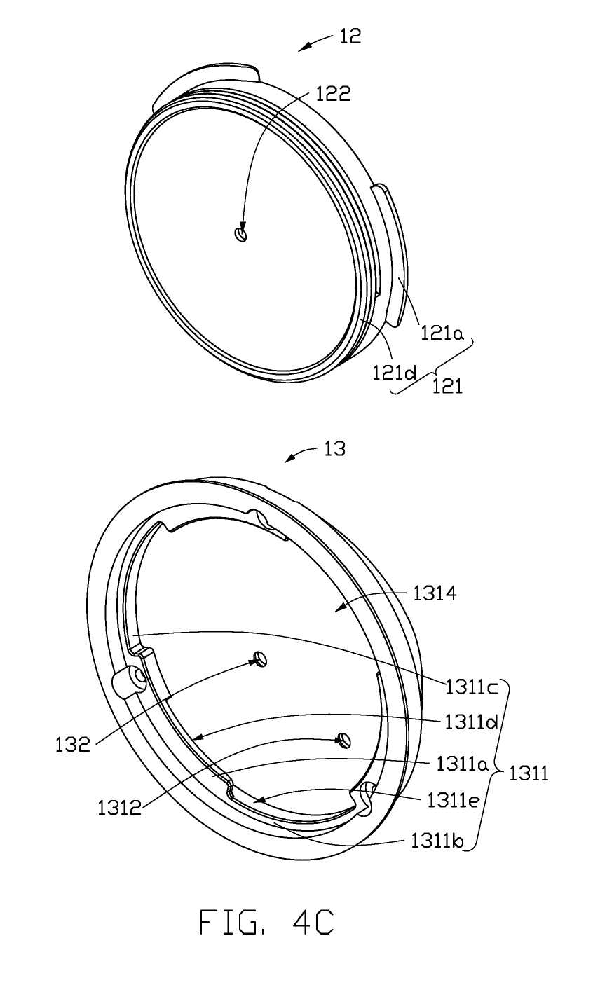

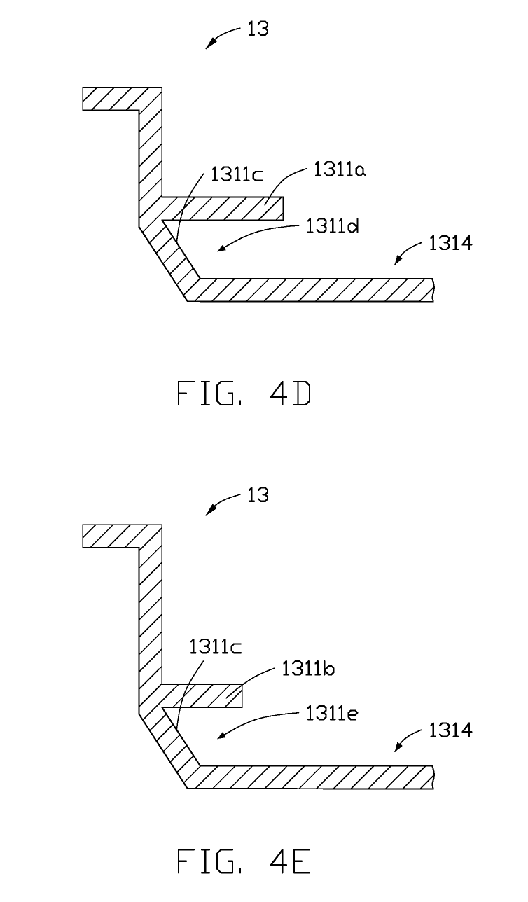

Referring to FIG. 4A, a front side view of the chest piece 12 and the chest piece holder 13 is provided in accordance with an exemplary embodiment of the present disclosure. The term “front side” refers to a side facing or toward the subject when conducting auscultation. The chest piece 12 comprises a dislocating structure 121 and a central hole 122. The dislocating structure 121 comprises a plurality of projections 121a being integral with and protruded outwardly from the chest piece 12. Each projection 121a is separate from another projection 121a. The chest piece holder 13 comprises a central hole 132 and a connecting structure 131. The connecting structure 131 comprises a flange 1311, a positioning aperture 1312, an indication 1313, and a concave space 1314 (not shown in FIG. 4A, but illustrated in FIG. 4C-4E). The flange 1311 is a flange structure extends inwardly on the front side of the chest piece holder 13, and comprises a plurality of wings 1311a and a plurality of notches 1311b. The wings 1311a are inwardly protruding pans of the flange 1311, and the notches 1311b are parts of the flange 1311 other than the wings 1311a. The notches 1311b and the wings 1311a are alternatingly arranged around the flange 1311. Therefore, each of the wing 1311a is next to one of the notch 1311b, and the amount of the wings 1311a and the notches 1311b is always the same. The amount of the notches 1311b and the projections 121a is also the same. There are at least 2 notches 1311b in the chest piece holder 13, and the amount of the notches 1311b and the projections 121a can be as many as they are structurally and geometrically plausible in the chest piece holder 13 and the chest piece 12. The concave space 1314 is defined by and surrounded by the flange 1311 on the front side of the chest piece holder 13, will be described further below.

Referring to FIG. 4B, a rear side view of the chest piece 12 and the chest piece holder 13 is provided in accordance with an exemplary embodiment of the present disclosure. The term “rear side” refers to a side that is opposite to the front side, and can be the side facing the user when conducting auscultation. The dislocating structure 121 of the chest piece 12 further comprises a plurality of positioning apertures 121b for aligning with the positioning aperture 1312, and an indication 121c on the rear side for aligning with the indication 1313 of the chest piece holder 13, this alignment makes an insert of the chest piece 12 into the chest piece holder 13 easier. The positioning aperture 121b comprises a positioning aperture 1211 and a positioning aperture 1212.

Referring FIG. 4C, a perspective view of the chest piece 12 and the chest piece holder 13 is provided in accordance with an exemplary embodiment of the present disclosure. The dislocating structure 121 of the chest piece 12 may further comprise a screw 121d on the side. The flange 1311 further comprises a sidewall 1311c. The concave space 1314 is structurally complementary to the chest piece 12.

Referring to FIG. 4D, a cross-sectional views of the wings 1311a of the chest piece holder 13 are provided in accordance with an exemplary embodiment of the present disclosure. The sidewall 1311c and the plurality of the wings 1311a form a plurality of wing receiving spaces 1311d at an edge of the concave space 1314. Referring to FIG. 4E, a cross-sectional views of the notches 1311b of the chest piece holder 13 are provided in accordance with an exemplary embodiment of the present disclosure. The notches 1311b is shorter than the wings 1311a. The sidewall 1311c and the plurality of the notches 1311b form a plurality of notch receiving spaces 1311e at another edge of the concave space 1314.

The chest piece 12 can be removably coupled to the chest piece holder 13. The dislocating structure 121 of the chest piece 12 can be removably coupled to the connecting structure 131 of the chest piece holder 13. Referring to FIGS. 5A and 5B, perspective views of a combination of the chest piece 12 and the chest piece holder 13 is provided in accordance with an exemplary embodiment of the present disclosure in FIGS. 5A and 5B, the rear side of the chest piece 12 contacts the front side of the chest piece holder 13.

FIG. 5A illustrates a first state of the digital auscultation device 10. The first state is defined by each of the projections 121a being received by each of the notch receiving spaces 1311e (shown on FIG. 4E). The height of the projection 121a is smaller than the height of the notch receiving space 1311e and the wing receiving space 1311d, therefore the projections 121a of the dislocating structure 121 generally lit the spaces formed by the sidewall 1311c, the notches 1311b, and the wings 1311a. Thus, the user is able to insert the chest piece 12 into the concave space 1314 of the chest piece holder 13. The indication 121c (not shown) of the chest piece 12 corresponds to the location of the indication 1313 (not shown) of the chest piece holder 13. With markings provided by the indication 121c and the indication 1313, the user may position the chest piece 12 and the chest piece holder 13 accurately. In the first state, the positioning aperture 1211 (not shown in FIG. 5A, but illustrated in FIG. 4B) on the rear side of the chest piece 12 matches the positioning aperture 1312 (not shown in FIG. 5A, but illustrated in FIG. 4A) on the front side of the chest piece holder 13. A ball (not shown) can be disposed in the positioning aperture 1312 to assist the matching between the positioning aperture 1211 and the positioning aperture 1312. In the first state, the ball is accommodated in both the positioning aperture 1211 and the positioning aperture 1312, therefore the user would be notified that the chest piece 12 is adequately received by the chest piece holder 13.

FIG. 5B illustrates a second state of the digital auscultation device 10. The second state is defined by each of the projections 121a being at least partially received by each of the wing receiving spaces 1311d (shown on FIG. 4D). The projection 121a can be partly or entirely received by the wing receiving space 1311d. In other words, a part of the projection 121a can be seen from the front side during the second state when the projection 121a is only partially received by the, wing receiving space 1311d. The central hole 122 of the chest piece 12 is located above the central hole 132 of the chest piece holder 13. In the second state, the indication 1313 of the chest piece holder 13 may be seen from the front side, and the positioning aperture 1212 (not shown on FIG. 5B, but illustrate in FIG. 4B) on the rear side of the chest piece 12 matches the positioning aperture 1312 (not shown in FIG. 5B, but illustrated in FIG. 4A) of the chest piece holder 13. The ball (not shown) disposed in the positioning aperture 1312 can assist the matching between the positioning aperture 1212 and the positioning aperture 1312, therefore the user would be notified that the chest piece 12 and the chest piece holder 13 is accurately coupled. The user may attach the diaphragm 11 onto the chest piece 12 to prepare for the auscultation.

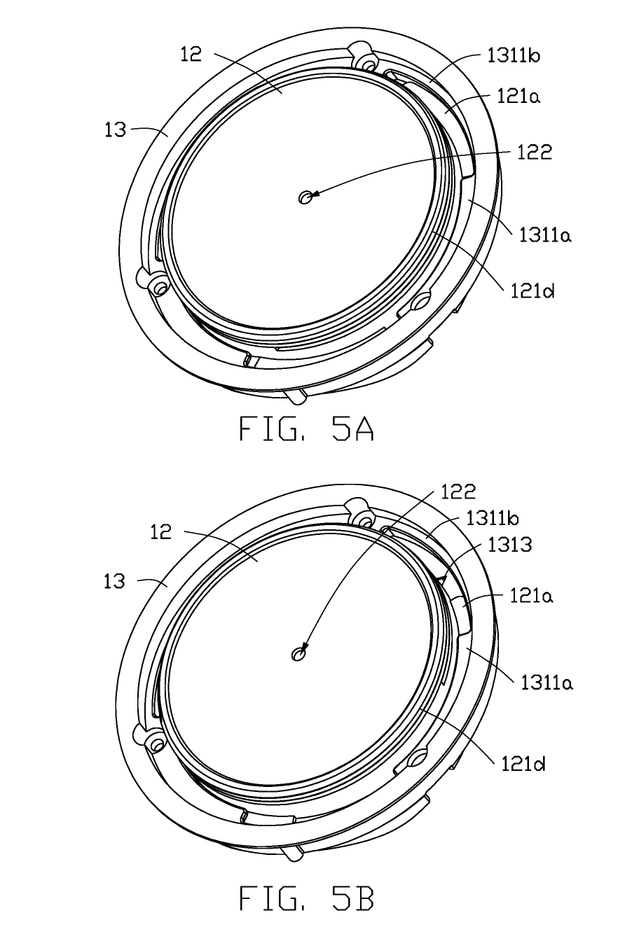

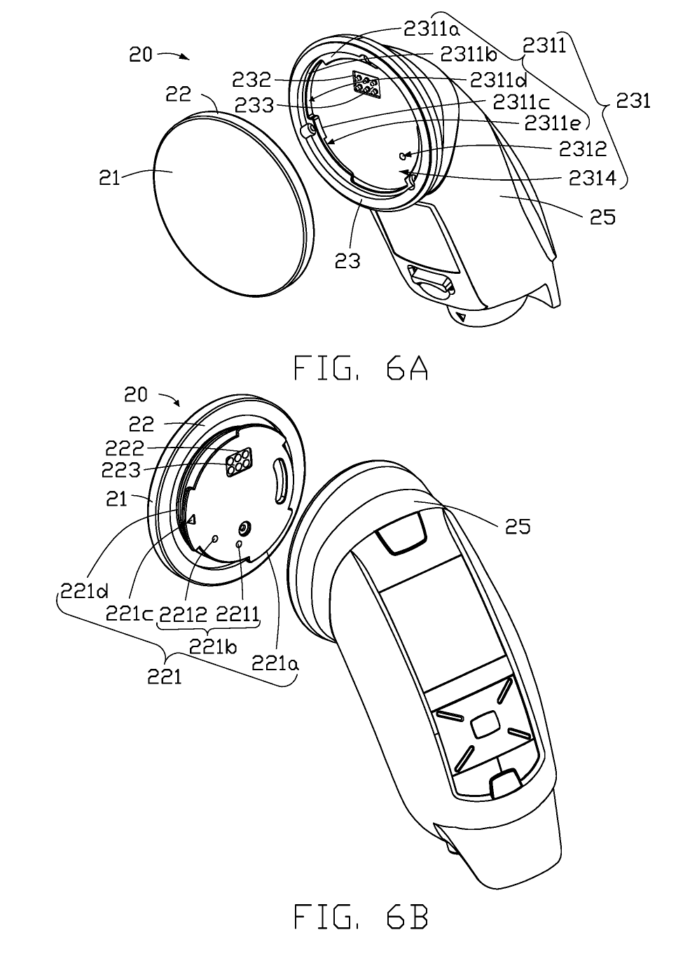

Referring to FIGS. 6A and 6B, perspective views of another digital auscultation device 20 is provided in accordance with an exemplary embodiment of the present disclosure. The digital, auscultation device 20 comprises a diaphragm 21, a chest piece 22, a chest piece holder 23, and a housing 25. In FIGS. 6A and 6B, the diaphragm 21 and the chest piece 22 are already coupled before the chest piece 22 being combined with the chest piece holder 23. The housing 25 comprises a control module (not shown) disposed in the housing 25. The chest piece 22 comprises a dislocating structure 221, a window 222, a microphone (not shown), a converter (not shown), and a pogo pin connector 223. The microphone is disposed in the chest piece 22, captures analog signals and transmit the analog signals to a converter. The converter is also disposed in the chest piece 22, converts the analog signals into digital signals and transmits the digital signals to the pogo pin connector 223. The dislocating structure 221 comprises a plurality of projections 221a protruding outwardly from the chest piece 22, a plurality of positioning apertures 221b, and a indication 221c extending rearwardly from the chest piece 22. The chest piece holder 23 comprises a connecting structure 231, a window 232 and a pogo pin 233. The pogo pin 233 is coupled to the control module in the housing 25. The connecting structure comprises a flange 2311 extending inwardly on the front side of the chest piece holder 23, a positioning aperture 2312, and a concave space 2314 defined by and surrounded by the flange 2311, wherein the flange 2311 comprises a plurality of wings 2311a, a plurality of notches 2311b, and a sidewall 2311c. The wings 2311a are inwardly protruding parts of the flange 2311, and the notches 2311b are parts of the flange 2311 other than the wings 2311a. The notches 2311b and the wings 2311a are alternatingly arranged around the flange 2311. Therefore, each of the wing 2311a is next to one of the notch 2311b, and the amount of the wings 2311a and the notches 2311b is always the same. A plurality of wing receiving spaces 2311d is formed by the wings 2311a and the sidewall 2311c, and a plurality of notch receiving spaces 2311e is thrilled by the notches 2311b and the sidewall 2311c.

The chest piece 22 can be removably coupled to the chest piece holder 23. The concave space 2314 and the rear side of the chest piece 22 is complementary to each other. The dislocating structure 221 can be removably coupled to the connecting structure 231. During a first state, each of the projections 221a is received by each of the notch receiving space 2311e, and the positioning aperture 2211 on the rear side of the chest piece 22 matches the positioning aperture 2312 on the front side of the chest piece holder 23. A ball (not shown) can be disposed in the positioning aperture 2312 to assist the matching between the positioning aperture 2211 and the positioning aperture 2312. In the first state, the ball is accommodated in both the positioning aperture 2211 and the positioning, aperture 2312.

During a second state, each of the projections 221a is at least partially received by each of the wing receiving space 2311d. When the window 222 matches the window 232, the pogo pin connector 223 matches the location of the pogo pin 233, thereby the digital signals is able to be transmitted from the pogo pin connector 222 to the pogo pin 233, and further be transmitted to the control module for analysis and recording. During the second state, the positioning aperture 2211 of the chest piece 22 matches the positioning aperture 2312 of the chest piece holder 23. The ball (not shown) disposed in the positioning aperture 2312 can assist the matching between the positioning aperature 2212 and the positioning aperature 2312, therefore the user would he notified that the chest piece 22 and the chest piece bolder 23 is accurately coupled. The coupling between the pogo pin connector 223 and the pogo pin 233, and the matching between the positioning aperture 2212 and the positioning aperture 2312 suggest the digital auscultation device 20 is readily usable.

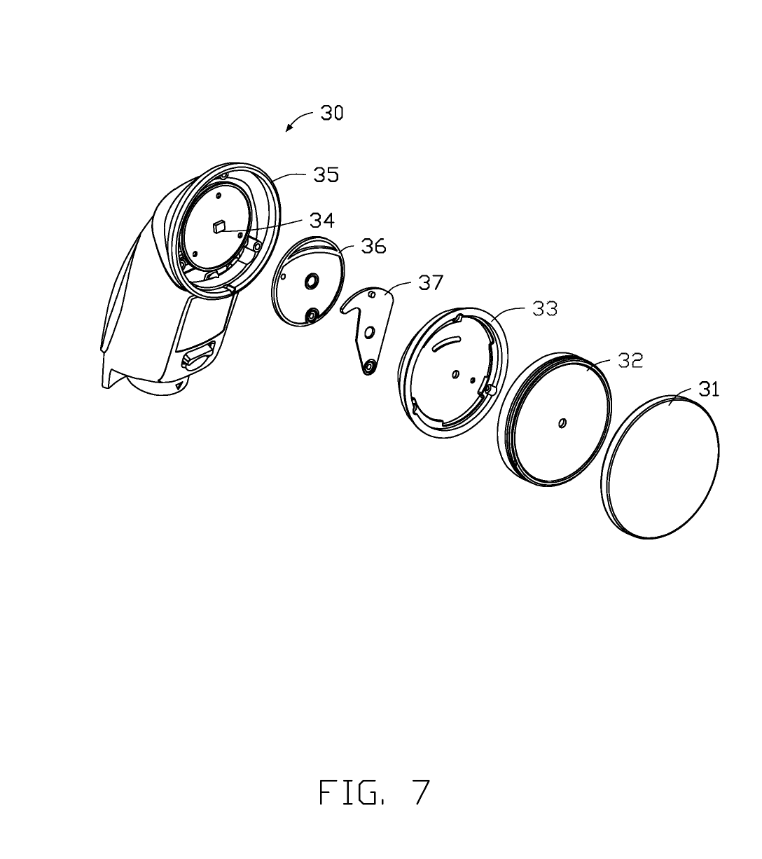

Referring to FIG. 7, an exploded view of a digital auscultation device 30 is provided in accordance with an exemplary embodiment of the present disclosure. The digital auscultation device 30 comprises a diaphragm 31, a chest piece 32, a chest piece holder 33, a microphone 34, a housing 35, a partition plate 36, and a switching piece 37. The microphone 34 is inserted in the housing 35 and facing the partition plate 36 on the trout side. The partition plate 36 and the switching piece 37 are disposed between the chest piece holder 33 and the microphone 34, and the partition plate 36 is between the switching piece 37 and the microphone 34. The chest piece 32 is removably coupled to the chest piece holder 33, and the diaphragm 31 is removably coupled to the chest piece 32.

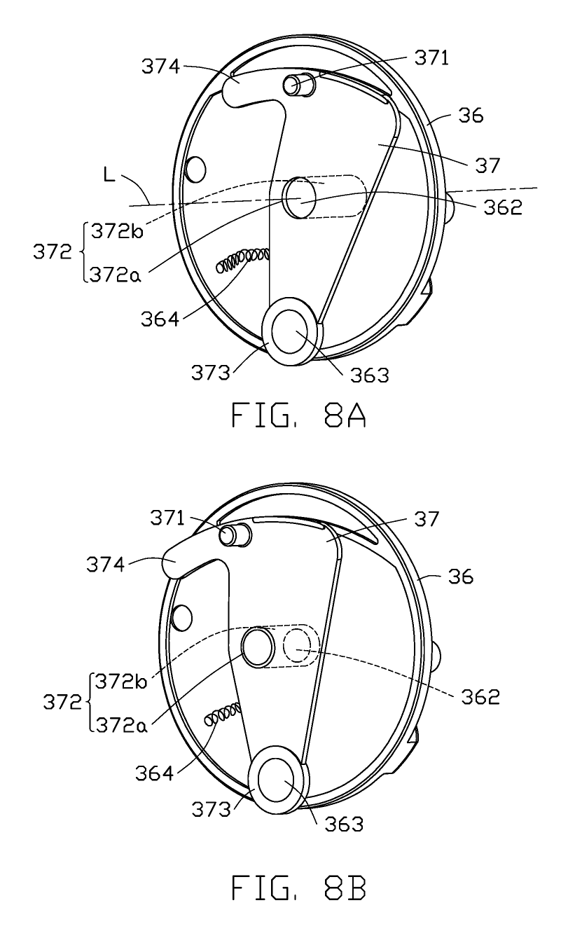

Referring to FIGS. 8A and 8B, perspective views of the partition plate 36 and the switching piece 37 are provided in accordance with an exemplary embodiment of the present disclosure. The partition plate 36 comprises a central hole 362 and an pivot 363. The central hole 362 corresponds to the position of the microphone 34 (not shown) of the digital auscultation device 30, and a sound transmission route L may pass through the central hole 362 to the microphone 34. The switching piece 37 comprises a protrusion 371, a door 372, an pivot-complementary structure 373, and a process 374. The protrusion 371 and the door 372 are rotatable about the pivot 363 of the partition plate 36. The door 372 comprise an opening 372a and a blocking portion 372b. The pivot-complementary structure 373 is coupled to the pivot 363, and the shape of the pivot-complementary structure 373 is complementary to the pivot 363. The pivot-complementary structure 373 can be a ring structure if the pivot 363 is a pole, whereas the pivot-complementary structure 373 can be a pole structure if the pivot 363 is an aperture or a recess.

FIG. 8A illustrates a third state of the partition plate 36 and the switching piece 37. The door 372 of the switching piece 37 does not block the central hole 362 of the partition plate 36. The opening 372a is corresponded to the central hole 362, and the sound transmission route L passes through the central hole 362 to the microphone 34 (not shown) is unblocked. The protrusion 371 is in a first position and the process 374 is in a third position during the third state of the switching piece 37 and partition plate 36. In the third state, a channel is formed by the opening 372a and the central hole 362, and the auscultation sound can pass through the opening 372a and the central hole 362 by air. The auscultation sound could come from the chest piece holder 33 (not shown), the chest piece 32 (not shown), or the diaphragm 31 (not shown).

FIG. 8B illustrates a fourth state of the partition plate 36 and the switching piece 37. The door 372 of the switching piece 37 at least partially or completely blocks the central hole 362 of the partition plate 36. The opening 372a does not correspond to the central hole 362 and is correspond to other portions of the partition plate 36. The protrusion 371 is in a second position and the process 374 is in a fourth position during the fourth state of the switching piece 37 and partition plate 36. The sound transmission route L passes through the central hole 362 to the microphone 34 (not shown) is at least partially or completely blocked by the blocking portion 372b of the door 372. In the fourth state, the auscultation sound would encounter obstacles, and the auscultation sound may not reach the microphone 34 by air.

A spring 364 may be disposed on the partition piece 36 and in contact with the door 372, The spring 364 is relaxed in the fourth state, and is stretched in the third state. During the fourth state, when the protrusion 372 is in a first position and the door 372 corresponds to the central hole 362, the spring 364 is stretched. During the third state, when the protrusion 372 is in a second position and the door 372 at least partially blocks the central hole 362, the spring 364 is relaxed. Therefore, the spring 364 is used to recover the position of the switching piece 37. The spring 364 can also be in contact with the process 374, or other portions of the switching piece 37 that is rotatable about the pivot 363.

Although configurations of the switching piece 37 and the partition plate 36 in FIGS. 8A and 8B are for the digital auscultation device, they could also be applied to non-digital auscultation devices. When these configuration applied in the non-digital auscultation device, the sound transmission route L passes through the central hole to a sound capturing element of the non-digital auscultation device in FIG. 8A, and the sound transmission route L is at least partially or completely blocked by the blocking portion 372b of the door 372 in FIG. 8B, therefore the auscultation sound may not reach the sound capturing element in the non-digital auscultation device.

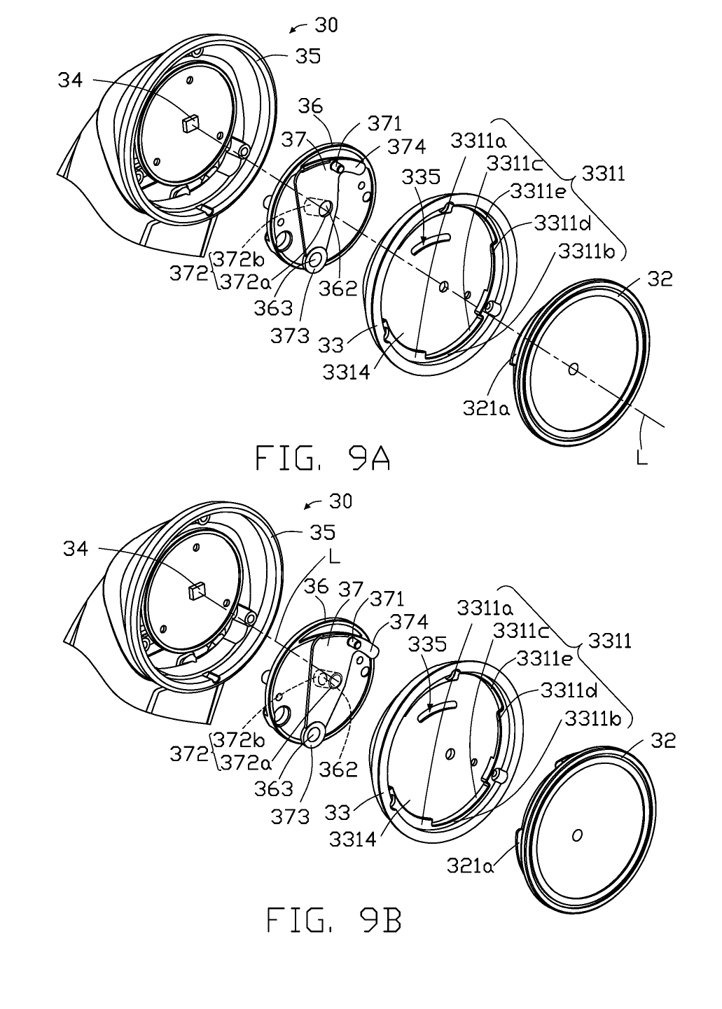

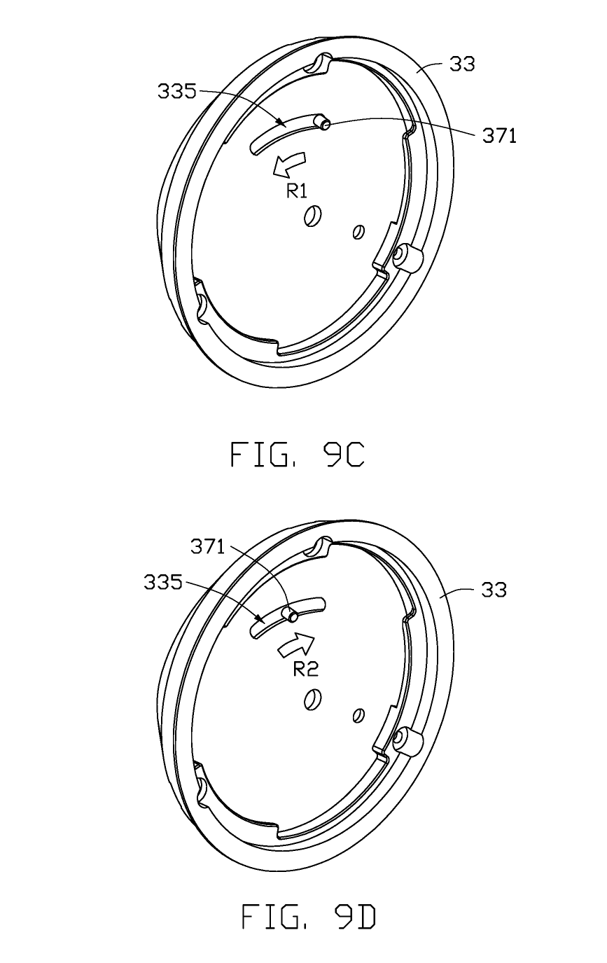

Referring to FIGS. 9A and 9B, exploded views of the digital auscultation device 30 are provided in accordance with an exemplary embodiment of the present disclosure. The chest piece 32 comprises a plurality of projections 321a protruding on the rear side. The chest piece holder 33 comprises a flange 3311, a concave space 3314, and a ditch 335. The flange 3311 comprises a plurality of wings 3311a protruding inwardly, a plurality of notches 3311b, and a sideman 3311c, The plurality of notches 3311b are parts on the flange 3311 that are not wings 3311a. According to similar configurations in FIGS. 4D and 4E, a plurality of wing receiving space 3311d are formed by the sidewall 3311c and the wings 3311a, and a plurality of notch receiving spaces 3311e are formed by the sidewall 3311c and the notches 3311b. The concave space 3314 is complementary to the rear side of the chest piece 32. The ditch 335 is an arc-shaped ditch.

FIG. 9A illustrates an exploded view of the second state of the digital stethoscope 30 and the third state of the partition plate 36 and the switching piece 37. In FIG. 9A, the distance between the chest piece 32, the chest piece holder 33, the switching piece 37, and the housing 35 are enlarged in order to demonstrate the spatial relationship between the components of the digital auscultation device 30 during the second state and the third state. The protrusion 371 is in a first position and corresponded within a location in the ditch 335. The projections 321a are disposed on a position that is corresponded to the wings 3311a. The sound transmission route L is established by combining a center of the chest piece 32, a center of the chest piece holder 33, the opening 372a, the central hole 362, and the microphone 34.

FIG. 9B illustrates an exploded view of the first state of the digital stethoscope 30 and the fourth state of the partition plate 36 and the switching piece 37. In FIG. 9B, the distance between the chest piece 32, the chest piece holder 33, the switching piece 37, and the housing 35 are enlarged in order to demonstrate the spatial relationship between the components of the digital auscultation device 30 during the first state and the third state. The protrusion 371 is in a second position and corresponded within another location in the ditch 335. The projections 321a are disposed on another position that is corresponded to the notches 3311b. The sound transmission route L is blocked by the blocking portion 372b of the switching piece 37, therefore the auscultation sound would be difficult to travel to the microphone 34 by air.

The protrusion 371 is in contact with the chest piece 32, and the rotation of the protrusion 371 is triggered by the rotation of the chest piece 32. Referring to FIG. 9C, a perspective view of the chest piece holder 33 and the switching piece 37 when in the fourth state, and the digital auscultation device 30 when in the first state is provided in accordance with an embodiment of the present disclosure. The protrusion 371 is in the second position within the ditch 335. The configuration of the switching piece 37 and the partition plate 36 in the fourth state provides a protective mechanism for the acoustic sensor during the changeover or replacement of the chest piece 32.

From FIG. 9C to 9D, a transformation from the first state to the second state of the digital auscultation device 30, and the fourth state to the third state of the switching piece 37 and the partition plate 36 is triggered by a rotation R1 of the switching piece 37. In FIG. 9C, the chest piece 32 (not shown) is in the first state wherein each of the projections 321a are received by each of the notch receiving, spaces 3311e (not shown). The chest piece 32 (not shown) is already in contact with the protrusion 371, and a rotation of the chest piece 32 triggers the rotation R1 of the protrusion 371 of the switching piece 37. The is of the chest piece 32 from FIG. 9C to 9D can be initiated by the user for coupling the chest piece 32 to the chest piece holder 33.

Referring to FIG. 9D, a perspective view of the chest piece holder 33 and the switching piece 37 when in the third state, and the digital auscultation device 30 when in the second state is provided in accordance with an embodiment of the present disclosure. The protrusion 371 is in the first position within the ditch 335. The sound transmission route L (as illustrated in FIG. 9B) is established and the auscultation sound can reach the microphone 34 by air. In FIG. 9D, the digital auscultation device 30 is in the second state, each of the projections 321a of the chest piece 32 is received by the wing receiving spaces 3311d. The configuration of the protections 321a and the wing receiving spaces 3311d during the second state of the digital auscultation device 30 are similar to the configuration of the projections 121a and the wing receiving spaces 1311d in FIG. 5B.

From FIG. 9D to 9C, a transformation from the second state to the first state of the digital auscultation device 30, and the third state to the fourth state of the switching piece 37 and the partition plate 36 is triggered by a rotation R2 of the switching piece 37. The rotation R2 of the protrusion 371 of the switching piece 37 is triggered by another rotation of the chest piece 32 (not shown). The rotation of the chest piece 32 from FIG. 9D to 9C can be initiated by the user for changing the chest piece 32 into another chest piece, or simply for uncoupling the chest piece 32.

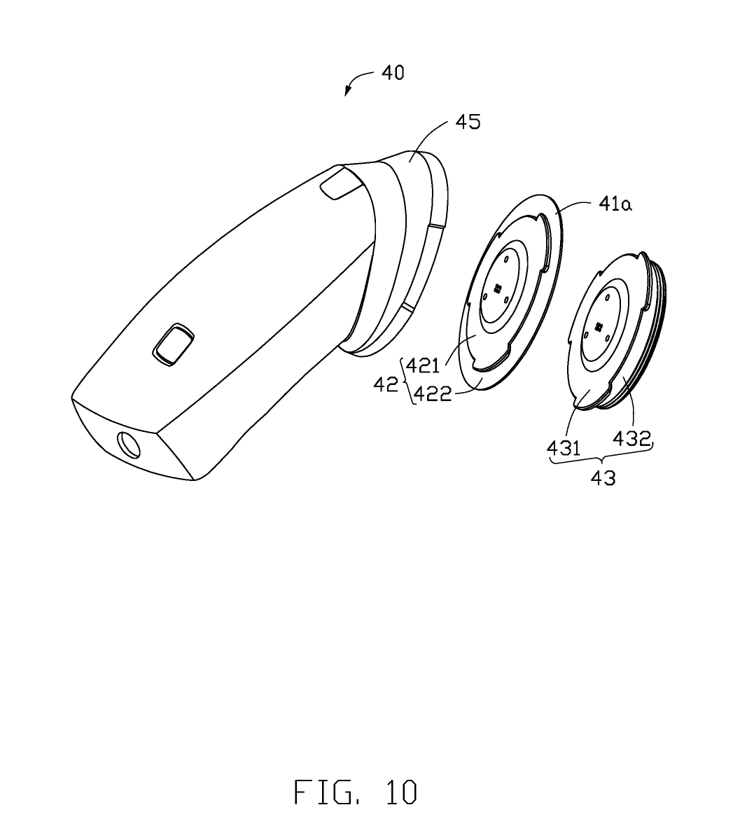

Referring to FIG. 10, an exploded view of a digital auscultation device 40 is provided in accordance with an exemplary embodiment of the present disclosure. The digital auscultation device 40 comprises a housing 45, a microphone (not shown), a chest piece holder (not shown), two interchangeable chest pieces 42 and 43. The chest piece holder of the digital auscultation device 40 is complementary to both the chest piece 42 and 43. The chest piece 42 comprises a platform 421 complementary to the chest piece holder of the digital auscultation device 40, and a diaphragm plane 422 for coupling with a diaphragm (not shown). The chest piece 43 comprises a platform 431 complementary to the chest piece holder of the digital auscultation device 40, and a diaphragm plane 432 for coupling with another diaphragm (not shown). The diaphragm of the chest piece 42 is larger than the diaphragm of the chest piece 43, in order for conducting auscultation on different subjects. For instance, the chest piece 41 is for pediatric auscultation because of the diaphragm of the chest piece 41 is smaller. The digital auscultation devices 10, 20, 30, and 40 of the present disclosure have removably-coupling configurations between the chest pieces and the chest piece holders, thus they are able to have chest pieces wherein the diaphragms on the chest pieces are of different sizes. The interchangeable chest piece design enables the digital auscultation device 10, 20, 30, and 40 of the present disclosure to be applied in different auscultation scenarios. The chest pieces and the diaphragms may have various sizes, geometry, material, or other structural configuration that may be beneficial to the operation or performance of the auscultation device under different circumstances.

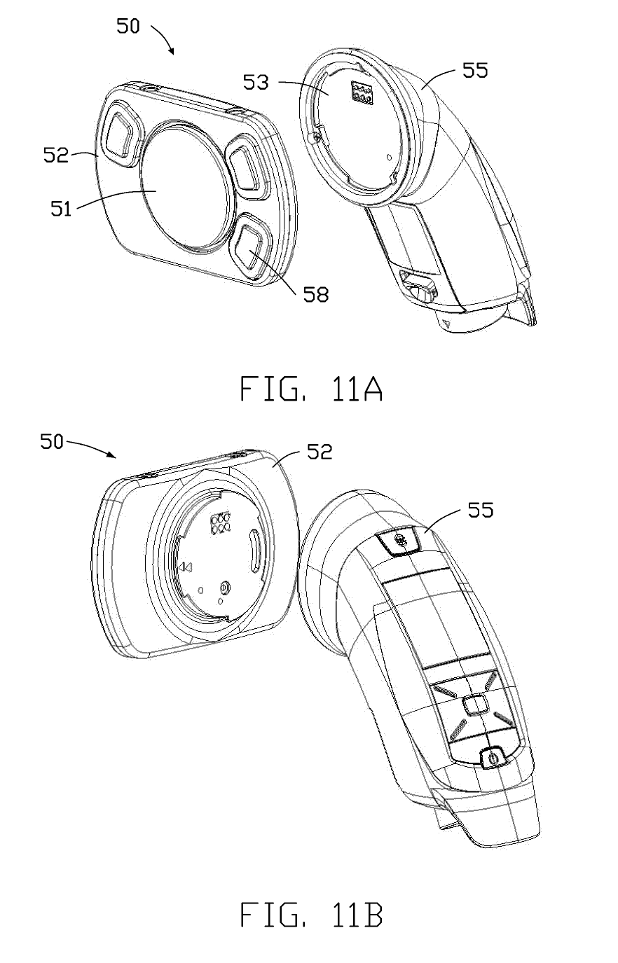

Referring to FIGS. 11A and 11B, perspective views of a digital auscultation device 50 are provided in accordance with an exemplary embodiment of the present disclosure. The digital auscultation device 50 comprises a diaphragm 51, a chest piece 52, a chest piece holder 53, a housing 55, and a plurality of ECG plates 58. The chest piece 52 can be removably coupled to the chest piece holder 53. The ECG plates 58 can be disposed on an extended surface on the front side of the chest piece 52, and is coupled with the chest piece 52 through lose logical coupling, tied physical coupling, or fully integrated with the chest piece 52. In FIG. 11A, the extended area is an area not covered by the diaphragm 51, or the auscultation module 51 when viewed from the front side. Multiple ECG plates 58 can be arranged corresponding to a lead arrangement in the conventional ECG instrument. In the digital auscultation device 50, two of the ECG plates 58 are disposed on one side of the chest piece 52 and one of the ECG plate 58 is disposed on another side of the chest piece 52. The digital auscultation device 50 is capable to conduct auscultation and ECG measurement at the same time, or switching between auscultation mode and ECG mode.

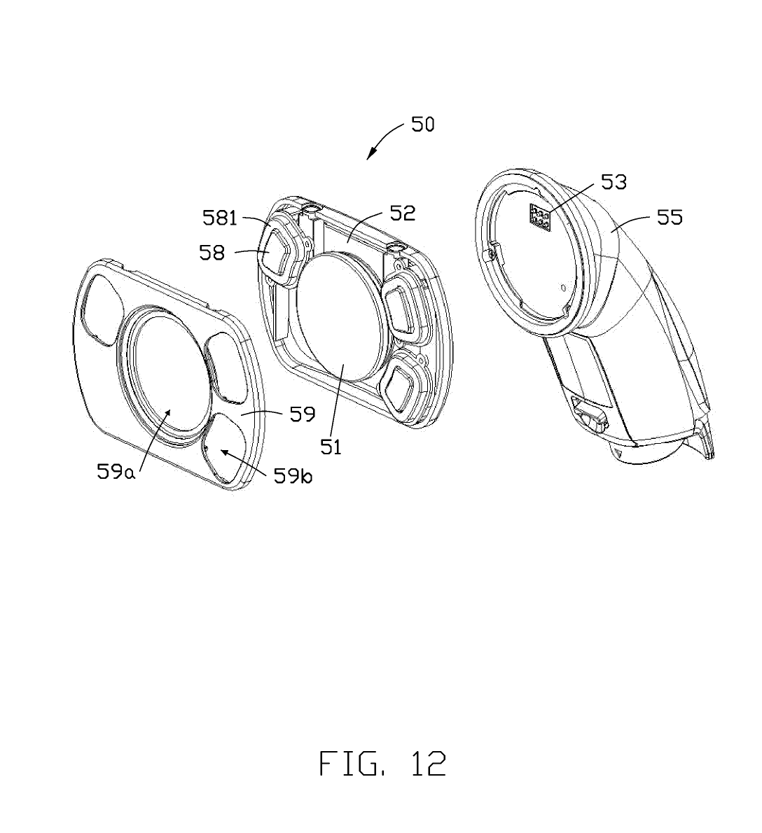

Referring to FIG. 12, an exploded view of the digital auscultation device 50 is provided in accordance with an embodiment of the present disclosure. The digital auscultation device 50 further comprises a cover 59 being coupled to the chest piece 52 on the front side. Because the ECG plates 58 measures electricity, therefore some circuits (not shown) may be arranged inside the chest piece 52 for transmitting the ECG signals captured by the ECG plates 58. The cover 59 protects the circuits from exposure, thus prevents short circuiting. The cover 59 comprises an opening 59a for the auscultation module 51, and a plurality of openings 59b for the ECG plates 58. The openings 59a and 59b conform the shape of the auscultation module 51 and the ECG plates 58, respectively. The ECG plates 58 may further comprise a rubber edge 581 for insulating the ECG plates 58 from the surrounding components, such as the cover 59 or the diaphragm 51.

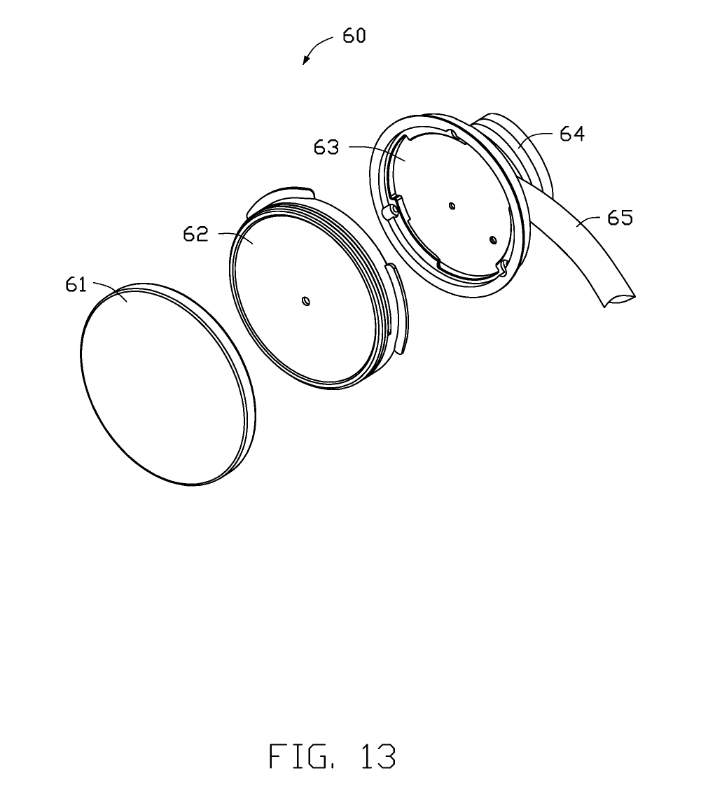

Referring to FIG. 13, an exploded view of a non-digital auscultation device 60 is provided in accordance with an exemplary embodiment of the present disclosure. The non-digital auscultation device 60 comprises a diaphragm 61, a chest piece 62, a chest piece holder 63, a bell 64, and a tube 65. When using the non-digital auscultation device 60, a distal end is closer to a surface of the auscultation subject, and a proximal end is closer to the user. The diaphragm 61 is removably coupled to the chest piece 62. The chest piece holder 63 is disposed on the distal end of the Don-digital auscultation device 60 and coupled to the bell 64, and the bell 64 is coupled to the tube 65. The bell 64 can be the sound capturing element of the non-digital auscultation device 60. The sound capturing element can also be a structure formed inside the non digital auscultation device that captures auscultation sounds. The chest piece 62 and the chest piece holder 63 can be removed, therefore the chest piece 62 in the non-digital auscultation device 60 is interchangeable. The removal and coupling, of the chest piece 62 and the chest piece holder 63 are similar to mechanisms illustrated in FIGS. 5A and 5B and respective descriptions therein. The configurations of the switching piece 37 and the partition plate 36 can be inserted between the chest piece holder 63 and the bell 64 for forming blocked or unblocked sound transmission routes.

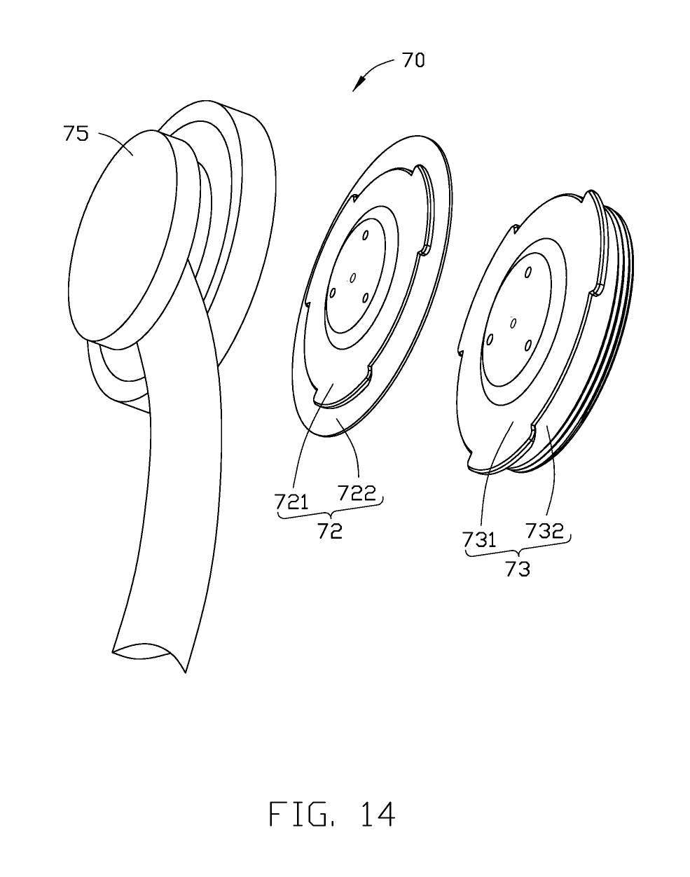

Referring to FIG. 14, an exploded view of a non-digital auscultation device 70 is provided in accordance with an embodiment of the present disclosure. The non-digital auscultation device 70 comprises a bell 75, a chest piece holder (not shown), two interchangeable chest pieces 72 and 73. The chest piece holder of the non-digital auscultation device 70 is complementary to both the chest piece 72 and 73. The chest piece 72 comprises a platform 721 complementary to the chest piece holder of the non-digital auscultation device 70, and a diaphragm plane 722 for coupling with a diaphragm (not shown). The chest piece 73 comprises a platform 731 complementary to the chest piece holder of the non-digital auscultation device 70, and a diaphragm plane 732 for coupling with a diaphragm (not shown). The diaphragm of the chest piece 72 is larger than the diaphragm of the chest piece 73, in order for conducting auscultation on different subjects. The interchangeable chest piece design enables the non-digital auscultation devices 60 and 70 of the present disclosure to be applied in different auscultation scenarios.

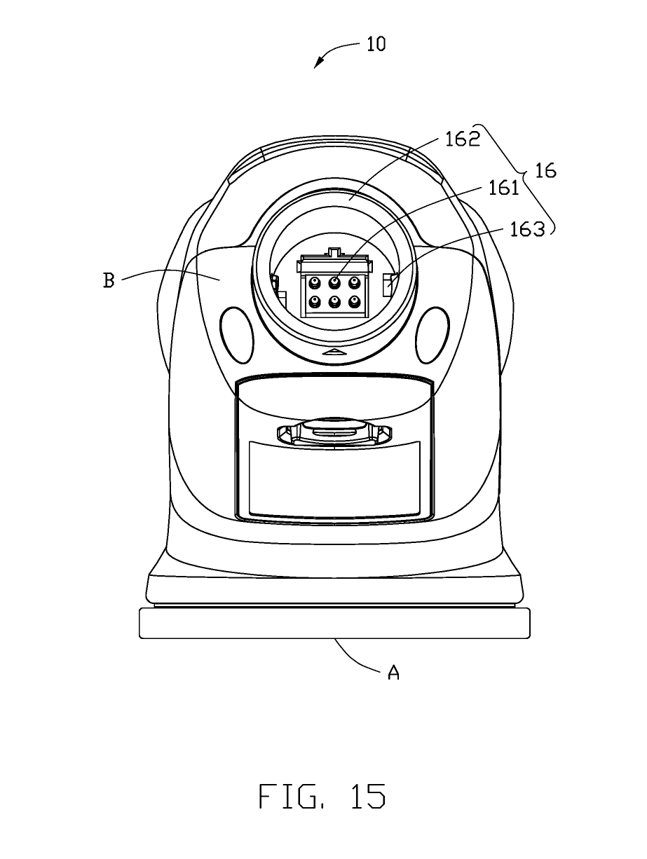

Referring to FIG. 15, a perspective view of the digital auscultation device 10 is provided in accordance with an embodiment of the present disclosure. The proximal part B of the digital auscultation device 10 is shown in FIG. 13. The digital auscultation device 10 comprises an output module 16 disposed on the proximal part B. The output module 16 can be connected to signal input components (not shown), and comprises a pogo pin connector 161 for electrically coupled to the signal input component and transferring the digital signals or other horn the digital auscultation device 10, a male screw thread 162 surrounding the pogo pin connector 161, and one or more latches 163 disposed in the inner side of the male screw thread 162.

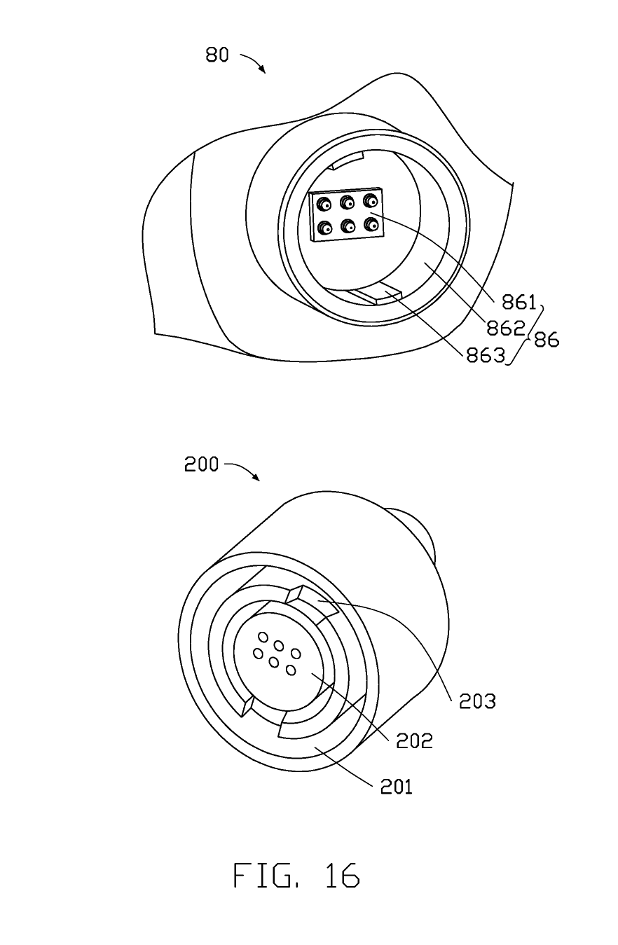

Referring to FIG. 16, a perspective view of a digital auscultation device 80 and a signal input component 200 is provided in accordance with an embodiment of the present disclosure. FIG. 16 illustrates only the proximal part B of the digital auscultation device 80 and a distal part of the signal input component 200. The digital auscultation device 80 comprises an output module 86 on the proximal part B, the output module 86 comprises a first pogo pin 861 for electrically coupled to the signal input component 200 and transferring the digital signals or other data from the digital auscultation device 80, a first male screw thread 862 surrounding the first pogo pins 61, and a first latch 863 disposed in the inner side of the first male screw thread 862. The signal input component 200 is an interface for transferring the digital signals or the auscultation results to other devices or to the user. The signal input component 200 comprises a first female screw thread 201, a first pogo pin connector 202 surrounded by the first female screw thread 201, and a first latch groove 203. The signal input component 200 may be a Y-tube, an audio output jack, a phone jack, or a wireless signal transmission component. When connecting with the signal input component 200, the first male screw thread 862 fits and is coupled with the first female screw thread 201 of the signal input component 200, the first pogo pin 861 is in contact with the first pogo pin connector 202 surrounded by the first female screw thread 201, and the first latch 863 is coupled to the first latch groove 203 of the signal input component 200. The output module 86 and the distal part of the signal input component 200 is complementary to each other. Therefore, alternative to configurations and elements in FIG. 16, a second male screw thread (not shown) could be a part of the signal input component 200 and a second female screw thread (not shown) could be a part of the output module 86; a second latch (not shown) could be a part of the signal input component 200 and a second latch groove (not shown) could be a part of the output module 86; a second pogo pin connector (not shown) could be a part of the signal input component 200 and a second pogo pin (not shown) could be a part of the output module 86.

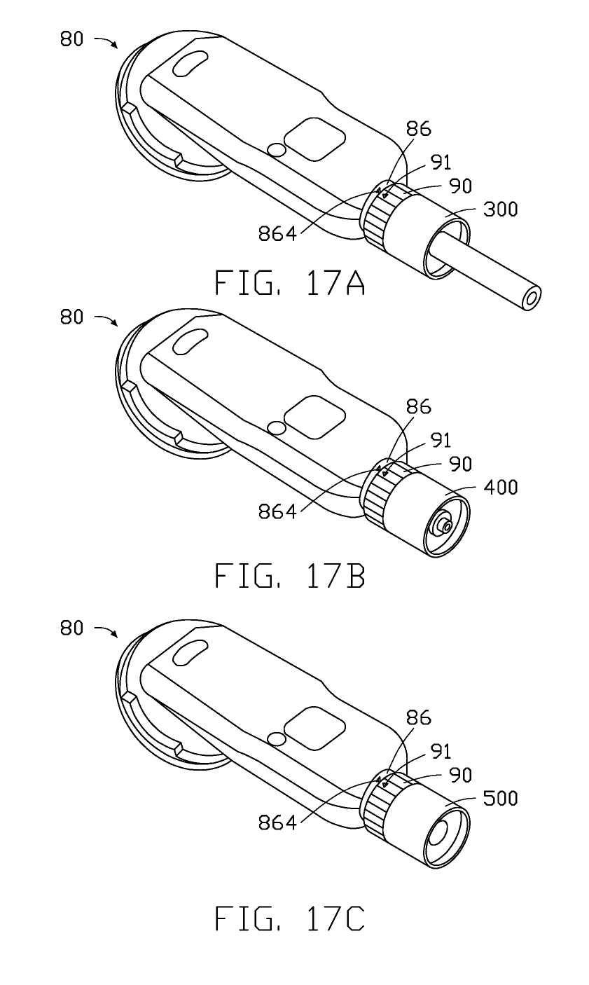

Referring to FIG. 17A-17C, perspective views of a plurality of modular digital auscultation devices 80 and various signal input components 300, 400, and 500 are provided in accordance with an embodiment of the present disclosure. With the output module 86, the modular digital auscultation device 80 is capable of working with and operatively connected to a variety of signal input components. The signal input components 200, 300, 400, and 500 are modules for transmitting the digital signals or the data to the user or other devices. The signal input components 200, 300, 400, and 500 can be Y-tube, an audio output jack, a phone jack, or a wireless signal transmission component. The wireless signal transmission component can be a Bluetooth dongle, a Wifi antenna or the like. The output module 66 further comprises a mark 864.

A rotary clasp 90 can be used to assist the respective coupling between the output module 86 and the signal input components 200, 300, 400, and 500. The rotary clasp 90 may cover at least a part of the output module 86, or the signal input components 200, 300, 400, and 500. The rotary clasp 90 comprises a mark 91 and an inner structure (not shown). The inner structure can be a clip or a thread. The rotary clasp 90 can assist the coupling when the signal input components 200, 300, 400, and 500 have complementary structures with respect to the output module 86, and can also assist the coupling when the signal input components 300, 400, or 500 lacks complementary structures with respect to the output module 86. For instance, if both the signal input component 500 and the output module 86 have male screw threads, they will not he able to be coupled; with the rotary clasp 90 between and combined with the two elements, the coupling of the output module 86 and the signal input component 500 is thus possible. The rotary clasp 90 may be coupled with the signal input component 200, 300, 400, or 500 first and then be coupled with the output module 86. The mark 91 is a marking for the user to accurately position the rotary clasp 90, the signal input component 200, 300, 400, or 500, and the output module 86. When coupling the rotary clasp 90 and the output module 86, the user would need to align the mark 91 of the rotary clasp 90 with the mark 864 of the output module 86. The rotary clasp 90 also could be a safety measure to prevent an accidental dislocation between the signal input components 200, 300, 400, or 500 and the output module 86. The accidental dislocation between the above components may occur if the subject has poor compliance during the auscultation. The user would have to unwind or un-clip the rotary clasp 90 on the modular digital auscultation device 80 before dislocating the output module 86 and the signal input components 200, 300, 400, or 500.

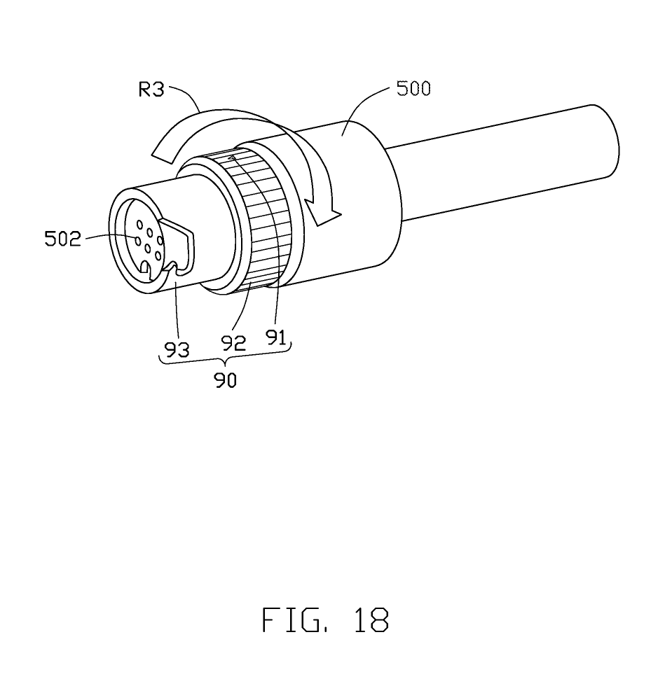

Referring to FIG. 18, a perspective view of the rotary clasp 90 and the signal input component 500 is provided in accordance with an exemplary embodiment of the present disclosure. The signal input component 500 comprises a pogo ping connector 502, and is already coupled with the rotary clasp 90. The rotary clasp 90 comprises the mark 91, a knob 92, and a sleeve 93. The sleeve 93 is a complementary structure to the output modules of the digital auscultation device 10, 20, 30, 40, 50, or 80. The sleeve 93 also covers the signal input component 500 and is able to assist a coupling between the signal input component 500 and the digital auscultation device 10, 20, 30, 40, 50, or 80. The coupling can be facilitated by the pogo pin connector 502 and the sleeve 93, a clip mechanism may be sufficient for the coupling between the sleeve 93 and the output modules. However, to uncouple the signal input component 500, the user would have to unwind the knob 91, and a rotation R3 is required to the knob 91. The knob 91 is a safety measure to prevent the accidental dislocation.

The embodiments shown and described above are only examples. Many details are often found in the art such as the other features of a digital auscultation device. Therefore, many such details are neither shown nor described. Even though numerous characteristics and advantages of the present technology have been set forth in the foregoing description, together with details of the structure and function of the present disclosure, the disclosure is illustrative only, and changes may he made in the detail, especially in matters of shape, size, and arrangement of the parts within the principles of the present disclosure, up to and including the full extent established by the broad general meaning of the terms used in the claims. It will therefore be appreciated that the embodiments described above may be modified within the scope of the claims.

What is claimed is:

1. An auscultation device, comprising;
a sound capturing element;
a chest piece holder disposed on a distal end of the auscultation device, coupled to the sound capturing element, and comprising a connecting structure; and
a chest piece comprising a dislocating structure removably coupled to the connecting structure of the chest piece holder.

2. The auscultation device of claim 1, wherein the dislocating structure comprises a plurality of projections; the connecting structure comprises a flange having a plurality of notches, a plurality of wings, and a sidewall; the notches and the wings are alternatingly arranged around the flange; the notches and the sidewall form a plurality of notch receiving spaces, and the wings and the sidewall form a plurality of wing receiving spaces.

3. The auscultation device of claim 2, wherein
each of the projections of the dislocating structure being received by each of the notch receiving spaces of the flange corresponds a first state of the auscultation device; and
each of the projections of the dislocating structure being at least partially received by each of the wing receiving spaces of the flange corresponds a second state of the auscultation device.

4. The auscultation device of claim 3, further comprising:
a partition plate between the sound capturing element and the chest piece holder, and comprising a central hole and a pivot; and
a switching piece between the partition plate and the chest piece holder, and comprising a door being rotatable about the pivot.

5. The auscultation device of claim 4, wherein
the door of the switching piece not blocking the central hole of the partition plate corresponds to a third state of the switching piece and the partition plate; and
the door of the switching piece at least partially blocking the central hole of the partition plate corresponds to a fourth state of the switching piece and the partition plate.

6. The auscultation device of claim 5, wherein the switching piece further comprises a protrusion: rotatable about the pivot, the chest piece holder further comprises a ditch, and the protrusion is in contact with the chest piece through the ditch.

7. The auscultation device of claim 6, wherein the auscultation device is in the third state when the protrusion of the switching piece is at a first position, and the auscultation device is in the fourth state when the protrusion of the switching piece is at a second position.

8. The auscultation device of claim 6, wherein
the auscultation device is in the first state when the switching piece and the partition plate are in the fourth state for at least partially blocking a sound transmission route from passing through the central hole to the sound capturing element;
a rotation of the switching piece is triggered by a rotation of the chest piece through the protrusion and transforms the switching piece and the partition plate from the fourth state into the third state; and
the auscultation device is in the second state when the switching piece and the partition plate are in the third state for unblocking the sound transmission route from passing through the central hole to the sound capturing element.

9. The auscultation device of claim 1, further comprising a diaphragm removably coupled to the chest piece, wherein the diaphragm can be of different sizes.

10. The auscultation device of claim 9, wherein the chest piece further comprises an extended surface not covered by the diaphragm, and one or more ECG plates disposed on the extended surface.

11. The auscultation device of claim 1, wherein the auscultation device further comprises:
a housing having a distal part and a proximal part;
a control module in the housing:
an output module connectable With a signal input component and disposed on the proximal part of the housing; and
wherein the sound capturing element coupled to the control module and is a microphone.

12. The auscultation device of claim 11 wherein the output module comprises a first latch, a first pogo pin, and a first male screw thread for connecting with different signal input components.

13. The auscultation device of claim 12, wherein each of the different signal input components comprises a first female screw thread, a first pogo pin connector, and a first latch groove.

14. The auscultation device of claim 11, further comprising a rotary clasp for connecting the signal input component and the output module.

15. The auscultation device of claim 11, wherein the output module comprises a second latch groove, a second pogo pin, and a second female screw thread for connecting with different signal input components.

16. The auscultation device of claim 15, wherein each of the different signal input components comprises a second male screw thread, a second pogo pin connector and a second latch.

17. The auscultation device of claim 11, wherein the signal input component is a Y-tube, an audio output jack, a phone jack, or a wireless signal transmission component.

18. A digital auscultation device, comprising:
a housing having a distal part and a proximal part;
a control module in the housing;
an output module connectable with an signal input component and disposed on the proximal part of the housing;
a chest piece holder disposed on the distal part of the housing and comprising a connecting structure; and
a chest piece comprising a dislocating structure removably coupled to the connecting structure of the chest piece holder.

19. The digital auscultation device of claim 18, wherein the chest piece further comprises an extended surface, and one or more ECG plates disposed on the extended surface.
